(19) United States
(12) Patent Application Publication
(10) Pub. No.: US 2025/0001140 A1
(43) Pub. Date: Jan. 2, 2025

(54) SPIRAL ENDOVASCULAR GUIDE WIRE

(71) Applicants: The United States Government as represented by the Department of Veterans Affairs, Washington, DC (US); The Board of Trustees of the University of Illinois, Urbana, IL (US)

(72) Inventor: Mladen Vidovich

(21) Appl. No.: 18/706,567

(22) PCT Filed: Nov. 1, 2022
(86) PCT No.: PCT/US2022/079044

§ 371 Date: May 1, 2024

Related U.S. Application Data
(60) Provisional application No. 63/274,701, filed on Nov. 2, 2021.

(51) Int. Cl. A61M 25/09; A61M 25/00; A61M 25/06

(52) U.S. Cl. CPC A61M 25/09; A61M 25/0026; A61M 25/0045; A61M 25/0606; A61M 2025/09075; A61M 2025/09166

(57) ABSTRACT

Provided herein is an endovascular guide wire comprising an elongated proximal section; a distal tip; and one or more intermediate sections disposed between the proximal section and distal tip. The one or more intermediate sections includes a three-dimensional spiral structure. The spiral structure can also be funnel-shaped. Also provided are systems include the endovascular guide wire and a catheter.

### CROSS-REFERENCE TO RELATED APPLICATIONS

This application claims benefit of and priority to U.S. Provisional Application No. 63/274,701, filed Nov. 2, 2021, the disclosure of which is incorporated herein by reference in its entirety.

### TECHNICAL FIELD

This invention relates to an endovascular guide wire comprising an elongated proximal section; a distal tip; and one or more intermediate sections disposed between said proximal section and distal tip, wherein said one or more intermediate sections comprises a three-dimensional spiral structure formed by said one or more intermediate sections.

### BACKGROUND

Endovascular guide wires are used in procedures in arteries or veins to investigate and treat vascular issues. These wires are routinely used in coronary, peripheral, and other endovascular interventions to deliver various equipment such as balloons, stents, and angioscopies.

For example, in typical angioplasty procedures, endovascular guide wires are used to navigate vessels to reach a lesion or vessel segment. Once the tip of the device arrives at its destination, an empty, collapsed balloon, known as a balloon catheter, is passed over a wire into the narrowed locations and then inflated to a fixed size. The balloon forces expansion of the stenosis (narrowing) within the vessel and the surrounding muscular wall, opening up the blood vessel for improved flow, and the balloon is then deflated and withdrawn. A stent may or may not be inserted at the time of ballooning to ensure the vessel remains open.

However, the stability and control of endovascular guide wires are still lacking. Therefore, there is a need for an endovascular guide wire with a spiral, three-dimensional structure that will increase stability and control.

### SUMMARY

Disclosed herein is an endovascular guide wire comprising an elongated proximal section; a distal tip; and one or more intermediate sections disposed between said proximal section and distal tip, wherein said one or more intermediate sections comprises a three-dimensional spiral structure formed by said one or more intermediate sections.

An embodiment of the present disclosure includes an endovascular guide wire that contains: an elongated proximal section; a distal tip; and one or more intermediate sections disposed between the proximal section and the distal tip. The intermediate section(s) can have a three-dimensional spiral structure formed by the intermediate section(s).

An embodiment of the present disclosure also includes a system including a catheter and an endovascular guide wire. The endovascular guide wire can include an elongated proximal section; a distal tip; and one or more intermediate sections disposed between the proximal section and the distal tip. The one or more intermediate sections comprises a three-dimensional spiral structure formed by the one or more intermediate sections.

Other compositions, apparatus, methods, features, and advantages will be or become apparent to one with skill in the art upon examination of the following drawings and detailed description. It is intended that all such additional compositions, apparatus, methods, features and advantages be included within this description, be within the scope of the present disclosure, and be protected by the accompanying claims.

### DESCRIPTION OF DRAWINGS

Further aspects of the present disclosure will be more readily appreciated upon review of the detailed description of its various embodiments, described below, when taken in conjunction with the accompanying drawings.

FIG. 1 is a schematic diagram of the intermediate section of an endovascular guide wire according to certain embodiments of the invention. The diagram shows the distal tip, which is the straight portion of the wire on the left side of the figure at the intersection of the axes, the intermediate section, which is a three-dimensional funnel around a straight centerline, and the proximal section, which is the straight portion on the right side of the figure.

FIG. 2 is a schematic diagram of the endovascular guide wire according to certain embodiments of the invention showing the intermediate section as a helical structure around a straight centerline where the radius remains constant at r and the distance between each revolution remains at the distance of P.

FIG. 3 is a schematic diagram of the endovascular guide wire according to certain embodiments of the invention showing the distal tip A and the intermediate section comprises a helical structure B with constant radius R1 which transitions to a helical structure C with constant radius R2 on a straight centerline.

FIG. 4 is a schematic diagram of the endovascular guide wire according to certain embodiments of the invention showing the intermediate section comprises an elongated helical structure A, a straight section B, and a tightly coiled helical structure C with constant radius on a straight centerline.

The drawings illustrate only example embodiments and are therefore not to be considered limiting of the scope described herein, as other equally effective embodiments are within the scope and spirit of this disclosure. The elements and features shown in the drawings are not necessarily drawn to scale, emphasis instead being placed upon clearly illustrating the principles of the embodiments. Additionally, certain dimensions may be exaggerated to help visually convey certain principles.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise.

As used herein, the following terms have the meanings ascribed to them unless specified otherwise. In this disclosure, “consisting essentially of” or “consists essentially” or the like, when applied to methods and compositions encompassed by the present disclosure refers to compositions like those disclosed herein, but which may contain additional structural groups, composition components or method steps (or analogs or derivatives thereof as discussed above). Such additional structural groups, composition components or method steps, etc., however, do not materially affect the basic and novel characteristic(s) of the compositions or methods, compared to those of the corresponding compositions or methods disclosed herein. “Consisting essentially of” or “consists essentially” or the like, when applied to methods and compositions encompassed by the present disclosure have the meaning ascribed in U.S. Patent law and the term is open-ended, allowing for the presence of more than that which is recited so long as basic or novel characteristics of that which is recited is not changed by the presence of more than that which is recited, but excludes prior art embodiments.

An endovascular guide wire is a medical device that is typically formed of a long, flexible metal wire having one or more components. They are generally used to gain access to a body structure or location by inserting it into the body and advancing it to the desired location. The endovascular guide wire can be used to probe, biopsy, penetrate, dilate or act as a vehicle for transporting an accompanying catheter to a given location. The current endovascular guide wires are lacking in stability and control which makes the delivery of devices in some anatomies challenging. The guide wires provided herein improve the stability and control needed to deliver devices in challenging anatomies.

Described herein is a surgical guide wire (e.g. an endovascular guidewire) comprising an intermediate section having a three-dimensional spiral structure during the insertion of the guide wire. Upon insertion of the intermediate section of the guide wire into a vessel, the three-dimensional spiral structure deflects the tip of the device off the wall of the vessel and makes the delivery of a device easier due to the stability and control created by the three-dimensional spiral structure. In certain embodiments, the intermediate section is about 10 to about 40 cm in length. In some embodiments, radio-opaque markers at the proximal and distal end of the intermediate section assist in orienting the intermediate section.

Endovascular guide wires for angioplasty, stent delivery, atherectomy and other intravascular procedures are known in the art. Such endovascular guide wires usually have an elongate core member with one or more segments near the distal end thereof which taper distally to smaller cross sections and terminate at a distal tip. The distal tip typically comprises a flexible rubber or plastic material. The tip is highly flexible and will not damage or perforate the vessel. The endovascular guide wire has enough flexibility to permit it to conform to curves or deviations present in the arteries. The endovascular guide wire preferably is not of the same rigidity along its entire length; the distal end is substantially more flexible to prevent the angioplasty wire from inadvertently puncturing the walls of the veins or arteries as it is being inserted. The portion behind the distal tip is increasingly stiff in order to better support a balloon catheter or similar device.

The majority of endovascular guide wires are straight. Abbott, however, has patented and sells the Hi-Torque Wiggle Guide Wire (U.S. Pat. No. 5,007,434). This endovascular guide wire angulated in a zig-zag fashion in a single plane to facilitate delivery of endovascular devices in tortuous vessels. The varied angles on the endovascular guide wire allow the tip of the device to deflect off the wall of the vessels and makes the delivery of the various equipment easier. However, the wire does not stabilize the vessel.

The angulations of the endovascular guide wire described herein stabilize the endovascular guide wire in the vessel and make it sturdier for device delivery. In addition to facilitating delivery in tortuous or calcified vessels, the coiled shape of the intermediate section can allow the coils to gently expand inside the vessel, exerting an outward radial force sufficient to create stability without tearing the vessel. Advantageously, the intermediate section can be used to stabilize the guide wire such that movement (such as a patient breathing or insertion of tools) does not cause the guide wire to shift. Advantageously, the coiled design may allow for improved tip attitude through tortuous vessels such as the coronary artery.

In some embodiments, the expanding force can be from about 1 atm to 30 atm, about 1 atm to 14 atm, about 1 atm to 12 atm, about 1 atm to 10 atm, or about 10 atm to 12 atm.

The endovascular guide wires described herein can be sized to suit various procedures. For example, the guide wire can be used in procedures such as neurosurgical, cardiac, urogynecological, nephrological, etc. Vessels, as used herein, can refer to any lumened structure in the body in which a guide wire may be used to insert a tool (e.g. balloon, scope, stent, ablation laser). Such vessels include, but are not limited to, veins, arteries, urethra, ureter, intestines, bile ducts, and the like.

As mentioned above, the overall length and diameter of endovascular guide wire may be varied to suit the particular procedures in which it is to be used and the materials from which it is constructed. The length of the endovascular guide wire generally ranges from about 65 cm to about 320 cm, or about 160 cm to about 200 cm. Endovascular guide wire diameters generally range from about 0.008 in. to about 0.035 in. (0.2 to 0.9 mm), or about 0.01 in. to about 0.018 in. (0.25 to 0.55 mm). Commercially available endovascular guide wires for coronary use are typically about 0.01, 0.012 and 0.014 inch (0.25, 0.3 and 0.036 mm) in diameter. In an embodiment, the endovascular guide wire described herein is about 0.007 to about 0.038 inch in diameter. The endovascular guide wire may be constructed of materials such as carbon-steel, titanium, Nitinol™, and beryllium-copper.

The endovascular guide wire may be covered by a polymer such as PTFE (TEFLON™), polyolefin, or polyurethane which can be bonded or otherwise tightly affixed to the core wire, and which itself has a low-friction surface, as is the case for TEFLON™, or whose surface can be coated with a low-friction surface. Other suitable coverings include a tube formed from virtually any polymer having exposed hydrogens, such as polyester, polyolefins, polycarbonate, polyvinylchloride, latex or silicon rubber, polystyrene, and polyacrylics, and a surface coating formed of a highly hydrophilic, low-friction polymer, such as polyvinylpyrrolidone (PVP), polyethyleneoxide, or polyhydroxyethylmethacrylate (polyHEMA) or copolymers thereof.

The endovascular guide wire may also comprise one or more topcoats of a silicone-based coating over the base coat. This combination of base coat and topcoat imparts a lubricity that improves the ability of the endovascular guide wire to overcome resistance and prevent lock up during use. Other coatings, such as hydrophobic, hydrophilic, or other antifouling coatings may be used.

Representative silicone-based coatings may comprise the components of the Microglide® coating used commercially by Guidant Corporation, amino functional dimethylsiloxane copolymer (Dow Corning MDX-4-4159 fluid, for example) and/or polydimethylsiloxane liquid (Dow 360, for example), and the like. Substrates for the silicone-based coating could be, as alternatives to TEFLON®, various fluoropolymers, polyethylene, polypropylene, stainless steel, or nickel titanium alloys.

The flexible polymer coating or sleeve can be applied to the endovascular guide wire by conventional polymer spraying or dipping methods, or by attaching a preformed polymer tube to the core segment(s). The latter can be accomplished by attaching the tube to the core under heat shrinking conditions, or by securing the tube to the endovascular guide wire by a suitable wire/polymer bonding agent. As indicated above, the lubricious surface coating may be formed by the surface of the covering, or preferably, by applying a lubricious polymer surface coating. Such a surface coating, which preferably covers a portion of the distal section, can be applied by spraying or dipping, according to known methods.

In each of the embodiments below, the intermediate section can have a radius (r) for each coil, a distance (p) between coils, and can contain a plurality of sections having different (r) and (p) from one another. As provided below, the intermediate sections can have clockwise coils, counterclockwise coils, or both. Although values and ranges of values are provided below, other values beneficial to specific surgical procedures can be envisioned by one of ordinary skill in the art. For example, the gauge of wire, coil direction, or other dimensions suitable for a for such as an arterial procedure, may be adjusted for a pulmonary or neurological procedure. The overall length of the guide wire and the placement of the intermediate section therein can vary depending on the procedure for which the guide wire may be used. For example, a shorter guide wire may be preferred for such as a ureter stent placement when compared to one for a cardiac procedure inserted through the femoral artery. The location of the intermediate section can be optimized based on the procedure being performed.

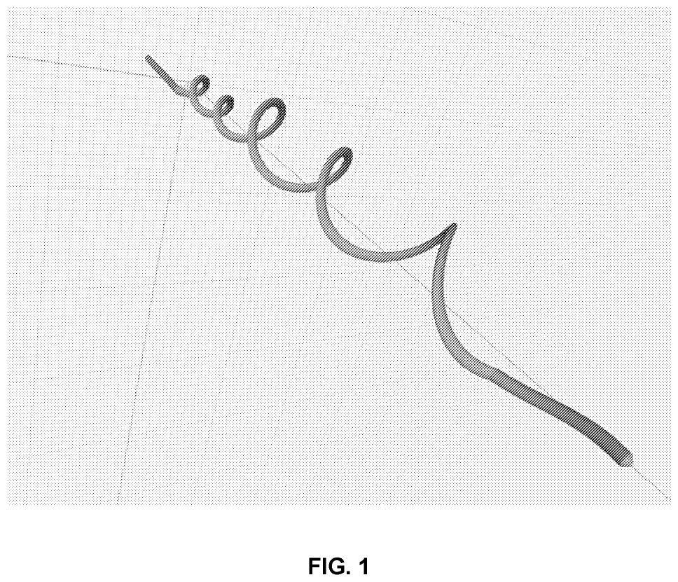

In certain configurations, the intermediate section of the endovascular guide wire will be oriented in a three-dimensional spiral around a straight centerline. The spiral structure formed by the endovascular guide wire itself can increase in diameter as it moves from the distal tip to the proximal section creating a funnel-shape (or coiled conical shape). FIG. 1 is a schematic diagram of the intermediate section according to certain embodiments of the invention showing the distal tip, which is the straight portion of the wire on the left side of the figure at the intersection of the axes, the intermediate section, which is the three-dimensional funnel around a straight centerline, and the proximal section, which is the straight portion on the right side of the figure. In an embodiment, the diameter of each singular loop of the endovascular guide wire around the straight centerline within the funnel-shape may increase from about 0.5 mm to 10 mm, about 0.5 mm to 60 mm, or about 0.5 mm to 100 mm. It can be appreciated that this spiral structure increases the stability and control while maneuvering through a vessel. A distance (p) between each of the loops may be about 0.5 mm to 5 mm, about 0.5 mm to 10 mm, or about 0.5 mm to 50 mm, and distance (p) may be constant or variable along the length of the funnel shape.

In certain configurations, the intermediate section of the endovascular guide wire will be oriented in a three-dimensional spiral around a straight centerline. The spiral structure formed by the endovascular guide wire itself can decrease in diameter as it moves from the distal tip to the proximal section creating a funnel-shape. In an embodiment, the diameter of each singular loop of the endovascular guide wire around the straight centerline within the funnel-shape may decrease from about 0.5 mm to 10 mm, about 0.5 mm to 60 mm, or about 0.5 mm to 100 mm.

In certain embodiments, the funnel-shape of the endovascular guide wire may be oriented in a clockwise direction. In another embodiment, the funnel-shape of the endovascular guide wire may be oriented in a counterclockwise direction. In still yet another embodiment, the intermediate section will be comprised of both clockwise sections and counterclockwise sections. It may be a clockwise section, straight line, and counterclockwise section. Alternatively, it may be a counterclockwise section, straight line, and clockwise section.

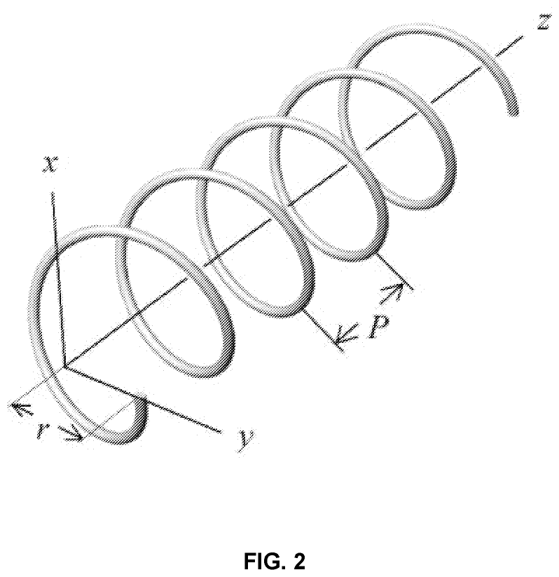

In certain configurations, the intermediate section of the endovascular guide wire formed by the endovascular guide wire itself will be oriented in a three-dimensional spiral around a straight centerline. The spiral structure can maintain its loop diameter as it moves from the distal tip to the proximal section creating a straight helical shape. FIG. 2 is a schematic diagram of the endovascular guide wire according to certain embodiments of the invention showing the intermediate section as a helical structure around a straight centerline, where the radius remains constant at r and the distance between each revolution remains at the distance of P. P can be from about 0.5 mm to 5 mm, about 0.5 mm to 10 mm, or about 0.5 mm to 50 mm. In an embodiment, the diameter of each singular loop around the straight centerline within the straight helical shape will remain between 0.5 mm to 5 mm, about 0.5 mm to 10 mm, or about 0.5 mm to 50 mm.

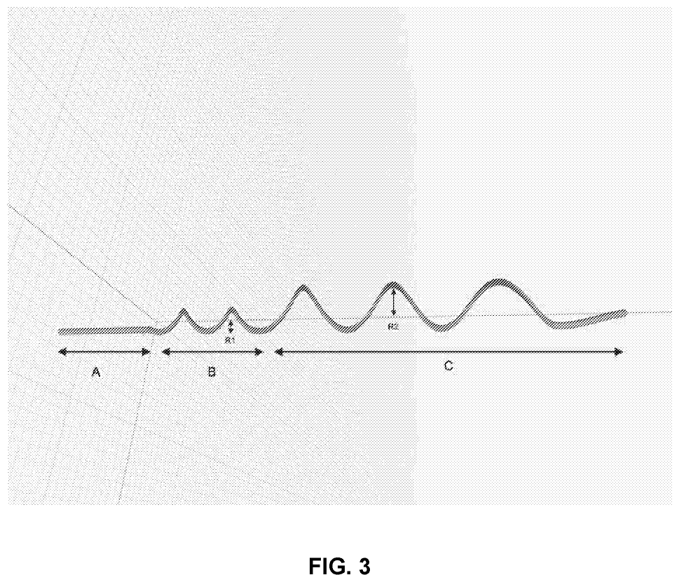

In certain embodiments, the straight helical shape formed by the endovascular guide wire itself may be oriented in a clockwise direction. In another embodiment, the straight helical shape formed by the endovascular guide wire itself may be oriented in a counterclockwise direction. In still yet another embodiment, the intermediate section will comprise both clockwise sections and counterclockwise sections. It may be a clockwise section, straight line, and counterclockwise section. Alternatively, it may be counterclockwise section, straight line, and clockwise section. One skilled in the art would appreciate that the number of configurations of the staggering between counterclockwise and clockwise can be limitless. Examples are FIG. 3 a schematic diagram of the endovascular guide wire according to certain embodiments of the invention showing the distal tip A and the intermediate section comprises a helical structure B with constant radius R1 which transitions to a helical structure C with constant radius R2 on a straight centerline. In other embodiments, the positions of the sections B and C could be reversed. Sections B and C can have lengths of about 1 mm to 100 mm and 1 mm to 100 mm, respectively.

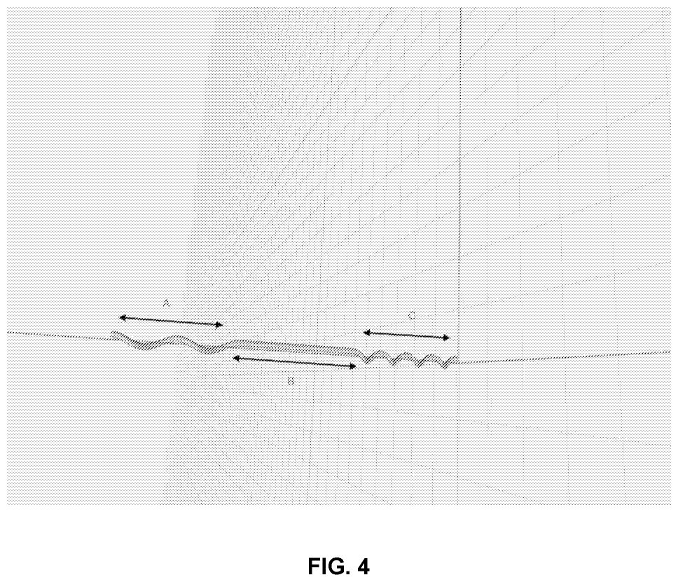

FIG. 4 provides a schematic diagram of the endovascular guide wire according to certain embodiments of the invention showing the intermediate section comprises an elongated helical structure A, a straight section B, and a tightly coiled helical structure C with constant radius on a straight centerline. In other embodiments, the positions of the more tightly coiled section C and elongated section A could be reversed. Sections A, B and C can have each have lengths of about 1 mm to 100 mm or about 1 mm to 50 mm or about 5 mm to 20 mm and can be the same or different from one another.

In certain configurations, the intermediate section of the endovascular guide wire will be oriented in a three-dimensional spiral around an articulating centerline. The spiral structure formed by the endovascular guide wire itself can increase in diameter as it moves from the distal tip to the proximal section creating a funnel-shape. In an embodiment, the diameter of each singular loop of the endovascular guide wire around the articulating centerline within the funnel-shape may increase from about 0.5 mm to about 10 mm. It can be appreciated that this spiral structure increases the stability and control while maneuvering through a vessel. Distance (p) can be from 0.5 mm to 5 mm, about 0.5 mm to 10 mm, or about 0.5 mm to 50 mm, or about 0.5 mm to 100 mm, and can be constant or variable along the spiral structure.

In certain configurations, the intermediate section of the endovascular guide wire will be oriented in a three-dimensional spiral around an articulating centerline. The spiral structure formed by the endovascular guide wire itself can decrease in diameter as it moves from the distal tip to the proximal section creating a funnel-shape. In an embodiment, the diameter of each singular loop of the endovascular guide around the articulating centerline within the funnel-shape may decrease from about 10 mm to about 0.5 mm. Distance (p) can be from about 0.5 mm to 10 mm, or about 0.5 mm to 50 mm, or about 0.5 mm to 100 mm, and can be constant or variable along the spiral structure.

In certain embodiments, the funnel-shape of the endovascular guide wire formed by the endovascular guide wire itself may be oriented in a clockwise direction. In another embodiment, the funnel-shape of the endovascular guide wire formed by the endovascular guide wire itself may be oriented in a counterclockwise direction. In still yet another embodiment, the intermediate section will comprise of both clockwise sections and counterclockwise sections. It may be a clockwise section, articulating line, and counterclockwise section. Alternatively, it may be counterclockwise section, articulating line, and clockwise section. One skilled in the art would appreciate that the number of configurations of the staggering between counterclockwise and clockwise can be limitless.

In certain configurations, the intermediate section of the endovascular guide wire formed by the endovascular guide wire itself will be oriented in a three-dimensional spiral around an articulating centerline. Articulating centerline refers to a centerline that may not be linear throughout the wire; the spiral section may loop around multiple centerlines along the length of the wire. The spiral structure can maintain its loop diameter as it moves from the distal tip to the proximal section creating a straight helical shape. In an embodiment, the diameter of each singular loop around the articulating centerline within the straight helical shape will remain at a diameter between about 0.5 mm to about 10 mm. Distance (p) can be from about 0.5 mm to 10 mm, or about 0.5 mm to 50 mm, or about 0.5 mm to 100 mm, and can be constant or variable along the spiral structure.

In certain embodiments, the straight helical shape formed by the endovascular guide wire itself may be oriented in a clockwise direction. In another embodiment, the straight helical shape formed by the endovascular guide wire itself may be oriented in a counterclockwise direction. In still yet another embodiment, the intermediate section will comprise of both clockwise sections and counterclockwise sections. It may be a clockwise section, articulating line, and counterclockwise section. Alternatively, it may be counterclockwise section, articulating line, and clockwise section. One skilled in the art would appreciate that the number of configurations of the staggering between counterclockwise and clockwise and be limitless.

In certain embodiments, the cross-sectional diameter of the one or more intermediate sections may be tapered and/or change along the length of the one or more intermediate sections. The cross-sectional diameter may vary from about 0.007 in. to about 0.037 in. In one embodiment, the cross-sectional diameter of an intermediate section may taper from about 0.008 in to 0.007 in moving towards the distal tip and then further taper within the distal tip to 0.003 in.

The endovascular guide wire of the invention further comprises radiopaque markers or bands placed at the proximate and distal ends of the one or more intermediate sections to aid in proper placement of the one or more intermediate sections within or outside of the blockage. The markers comprise one or more radiopaque materials such as platinum iridium, platinum nickel or platinum tungsten platinum, gold or a platinum/gold alloy. The highly radiopaque markers provide clear, visible fluoroscopic indication of the location of the one or more intermediate sections of the angioplasty wire to indicate clearly the endovascular guide wire position. In other embodiments, the endovascular guide wire can have a radiopaque coating along the entire wire or on the distal tip.

In another aspect, this invention is a kit comprising an endovascular guide wire comprising an elongated proximal section; a distal tip; and one or more intermediate sections disposed between said proximal section and distal tip, wherein said one or more intermediate sections comprises a three-dimensional spiral structure formed by said one or more intermediate sections.

One of skill in the art will appreciate that the endovascular guide wire of the invention is useful in a variety of procedures and with a variety of devices for treating blockages.

For example, in typical PTCA procedures endovascular guide wire of the invention is percutaneously introduced into the cardiovascular system of a patient in a conventional Seldinger technique and advanced therein until the radiopaque markers proper placement of the one or more intermediate section in the lesion to be dilated. A dilatation catheter having an inflatable balloon on the distal portion thereof is then advanced into the patient's coronary anatomy over the previously introduced endovascular guide wire until the balloon of the dilatation catheter is properly positioned across the lesion. Once in position across the lesion, the balloon is inflated to a predetermined size with radiopaque liquid at relatively high pressures (e.g. greater than 4 atmospheres) to compress the arteriosclerotic plaque of the lesion against the inside of the artery wall and to otherwise expand the inner lumen of the artery. The balloon is then deflated so that blood flow is resumed through the dilated artery and the dilatation catheter can be removed therefrom.

In some embodiments, the guide wire may be included in a dual lumen (rapid exchange) or over-the-wire system, as can be envisioned by one of ordinary skill in the art. For example, the wire could be provided alone or pre-loaded on a rapid exchange, over the wire or dual/multiple lumen catheter to facilitate advancement across diseased segments. A microcatheter assembly of the guide wire can specifically facilitate performance of a procedure.

In a particular embodiment, the guide wire provides stability in a catheter rather than directly in a vessel.

The intermediate section can be positioned to provide support in a location other than the surgical target. In a particular example, the intermediate portion can be sited in the arm of a patient during a coronary procedure to provide an anchored position for the wire such that a tool can be delivered through the brachial artery to the heart.

### Aspects of the Disclosure

The present disclosure will be better understood upon reading the following numbered aspects, which should not be confused with the claims. Any of the numbered aspects below can, in some instances, be combined with aspects described elsewhere in this disclosure and such combinations are intended to form part of the disclosure.

Aspect 1. An endovascular guide wire comprising: an elongated proximal section; a distal tip; and one or more intermediate sections disposed between the proximal section and the distal tip, wherein the one or more intermediate sections comprises a three-dimensional spiral structure formed by the one or more intermediate sections.
Aspect 2. The endovascular guide wire of aspect 1, wherein the one or more intermediate sections comprises a clockwise three-dimensional spiral structure or a counterclockwise three-dimensional spiral structure.
Aspect 3. The endovascular guide wire of aspects 1 or 2, wherein the three-dimensional spiral structure has a diameter of about 0.5 mm to 10 mm around a straight centerline.
Aspect 4. The endovascular guide wire of aspects 1 or 2, wherein the three-dimensional spiral structure has a diameter of about 0.5 mm to 10 mm around an articulating centerline.
Aspect 5. The endovascular guide wire of any of aspects 1-4, wherein the one or more intermediate sections comprises both clockwise three-dimensional spiral structures and counterclockwise three-dimensional spiral structures in series.
Aspect 6. The endovascular guide wire of any of aspects 1-5, having a diameter of about 0.007 in. to about 0.038 in.
Aspect 7. The endovascular guide wire of any of aspects 1-5, having a diameter that may vary over the one or more intermediate sections from about 0.007 in. to about 0.038 in.
Aspect 8. The endovascular guide wire of any of aspects 1-7, further comprising radio-opaque markers disposed at the proximal and distal end of the one or more intermediate sections.
Aspect 9. The endovascular guide wire of any of aspects 1-8, wherein the three-dimensional spiral structure is funnel shaped.
Aspect 10. A system comprising a catheter and an endovascular guide wire, the endovascular guide wire comprising: an elongated proximal section; a distal tip; and one or more intermediate sections disposed between the proximal section and the distal tip, wherein the one or more intermediate sections comprises a three-dimensional spiral structure formed by the one or more intermediate sections.
Aspect 11. The system of aspect 10, wherein the catheter is a dual lumen catheter or and over-the-wire catheter system.
Aspect 12. The system of aspects 10 or 11, where the endovascular guide wire is preloaded onto the catheter.

Although embodiments have been described herein in detail, the descriptions are by way of example. The features of the embodiments described herein are representative and, in alternative embodiments, certain features and elements may be added or omitted. Additionally, modifications to aspects of the embodiments described herein may be made by those skilled in the art without departing from the spirit and scope of the present invention defined in the following claims, the scope of which are to be accorded the broadest interpretation so as to encompass modifications and equivalent structures.

Disjunctive language such as the phrase “at least one of X, Y, or Z,” unless specifically stated otherwise, is otherwise understood with the context as used in general to present that an item, term, etc., may be either X, Y, or Z, or any combination thereof (e.g., X, Y, and/or Z). Thus, such disjunctive language is not generally intended to, and should not, imply that certain embodiments require at least one of X, at least one of Y, or at least one of Z to each be present.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

It should be noted that measurements, amounts, and other numerical data can be expressed herein in a range format. It is also understood that there are a number of values disclosed herein, and that each value is also herein disclosed as “approximately” that particular value in addition to the value itself. For example, if the value “10” is disclosed, then “approximately 10” is also disclosed. Similarly, when values are expressed as approximations, by use of the antecedent “approximately,” it will be understood that the particular value forms a further aspect. For example, if the value “approximately 10” is disclosed, then “10” is also disclosed.

As used herein, the terms “about,” “approximately,” “at or about,” and “substantially equal” can mean that the amount or value in question can be the exact value or a value that provides equivalent results or effects as recited in the claims or taught herein. That is, it is understood that amounts, sizes, measurements, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In general, an amount, size, measurement, parameter or other quantity or characteristic is “about,” “approximate,” “at or about,” or “substantially equal” whether or not expressly stated to be such. It is understood that where “about,” “approximately,” “at or about,” or “substantially equal” is used before a quantitative value, the parameter also includes the specific quantitative value itself, unless specifically stated otherwise.

Where a range is expressed, a further aspect includes from the one particular value and/or to the other particular value. Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y′, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y′, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

1. An endovascular guide wire comprising: an elongated proximal section: a distal tip; and one or more intermediate sections disposed between the proximal section and the distal tip, wherein the one or more intermediate sections comprises a three-dimensional spiral structure formed by the one or more intermediate sections.

2. The endovascular guide wire of claim 1, wherein the one or more intermediate sections comprises a clockwise three-dimensional spiral structure or a counterclockwise three-dimensional spiral structure.

3. The endovascular guide wire of claim 2, wherein the three-dimensional spiral structure has a diameter of about 0.5 mm to 10 mm around a straight centerline.

4. The endovascular guide wire of claim 2, wherein the three-dimensional spiral structure has a diameter of about 0.5 mm to 10 mm around an articulating centerline.

5. The endovascular guide wire of claim 1, wherein the one or more intermediate sections comprises both clockwise three-dimensional spiral structures and counterclockwise three-dimensional spiral structures in series.

6. The endovascular guide wire of claim 1, having a diameter of about 0.007 in. to about 0.038 in.

7. The endovascular guide wire of claim 1, having a diameter that varies over the one or more intermediate sections from about 0.007 in. to about 0.038 in.

8. The endovascular guide wire of claim 1, further comprising radio-opaque markers disposed at the proximal and distal end of the one or more intermediate sections.

9. The endovascular guide wire of claim 1, wherein the three-dimensional spiral structure is funnel shaped.

10. A system comprising a catheter and an endovascular guide wire, the endovascular guide wire comprising: an elongated proximal section: a distal tip; and one or more intermediate sections disposed between the proximal section and the distal tip, wherein the one or more intermediate sections comprises a three-dimensional spiral structure formed by the one or more intermediate sections.

11. The system of claim 10, wherein the catheter is a dual lumen catheter or and over-the-wire catheter system.

12. The system of claim 10, where the endovascular guide wire is preloaded onto the catheter.

13. The endovascular guide wire of claim 1, further comprising a polymer coating on at least a portion of a surface thereof.

14. The endovascular guide wire of claim 13, further comprising a silicone-based coating over at least a portion of the polymer coating.

15. The endovascular guide wire of claim 1, wherein the three-dimensional spiral structure formed by the one or more intermediate sections increases in diameter from the distal tip to the proximal section in a coiled conical shape.

16. A method for treating a neurological, cardiac, urogynecological, or nephrological condition in a subject, comprising advancing the endovascular guide wire of claim 1 into a lumenated structure of the subject.

17. The method of claim 16, wherein the lumenated structure of the subject is a vein, artery, urethra, ureter, intestine, or bile duct.

18. The method of claim 16, which is performed in a percutaneous transluminal coronary angioplasty (PTCA) procedure in which the endovascular guide wire of claim 1 is advanced into an artery of the subject and the artery wall is expanded.

19. The method of claim 16, wherein the three-dimensional spiral structure formed by the one or more intermediate sections of the endovascular guide wire of claim 1 increases in diameter from the distal tip to the proximal section in a coiled conical shape.

20. The system of claim 10, wherein the three-dimensional spiral structure formed by the one or more intermediate sections increases in diameter from the distal tip to the proximal section in a coiled conical shape.
